(19) United States
(12) Patent Application Publication
(10) Pub. No.: US 2009/0098171 A1
(43) Pub. Date: Apr. 16, 2009

(54) Cosmetic composition in the form of an emulsion comprising a continuous aqueous phase and a dispersed fatty phase, and method for its preparation

(75) Inventors: Valerie Alard, Orleans (FR); Dominique Quiles, Chemault (FR); Marc Dumas, Saint Jean le Blanc (FR); Eric Perrier, Les Cotes D'Arey (FR)

Correspondence Address: HAMRE, SCHUMANN, MUELLER & LARSON, P.C., P.O. BOX 2902, MINNEAPOLIS, MN, 55402-0902, US

(73) Assignee: LVMH RECHERCHE, Saint Jean de Braye (FR)

(21) Appl. No.: 12/284,861

(22) Filed: Sep. 25, 2008

(30) Foreign Application Priority Data
Sep. 26, 2007 (FR) 0706749

(51) Int. Cl. A61K 8/06; A61K 9/127

(52) U.S. Cl. 424/401; 424/450; 424/62

(57) ABSTRACT

The invention relates to a composition in the form of an emulsion comprising a continuous aqueous phase and a dispersed fatty phase, characterized in that said aqueous phase contains liposomes in the presence of a non-ionic surfactant chosen from fatty alcohol polyethylene glycol ethers and fatty alcohol polypropylene glycol ethers, and mixtures thereof, and of at least one polysaccharide in water-soluble form.

It also relates to a method for preparing this composition.

It also relates to a stable dispersion of liposomes that can be used for the preparation of the aqueous phase of this composition.

The invention relates to a cosmetic composition in the form of an emulsion comprising a continuous aqueous phase containing liposomes, and a dispersed fatty phase, and to the method for the preparation thereof.

Liposomes are unilamellar vesicles, i.e. vesicles made up of a single bilayer of amphiphilic lipids forming the envelope, or multilamellar vesicles, i.e. vesicles made up of several superimposed bilayers.

The structure of amphiphilic lipids, which are made up of a hydrophilic head and hydrophobic groups, and their arrangement in the bilayers, make possible the creation of hydrophilic or hydrophobic compartments within these bilayers.

A well-known drawback of liposomes is their sensitivity to the composition of the medium in which they are dispersed, and especially to the presence of compounds capable of degrading them, in particular the surfactants added to dispersed systems such as emulsions, in order to improve the stability thereof.

Cosmetic compositions in the form of an oil-in-water emulsion usually have a pleasant texture without inducing any drying out of it.

For this reason, the incorporation of liposomes into the continuous phase of these oil-in-water emulsions would allow those skilled in the art to develop different textures for the cosmetic compositions thus prepared, for example creams.

In addition, liposomes are also of great value in cosmetic applications for vectorizing active agents in the epidermis.

However, the sensitivity of liposomes to the presence of surfactants in the continuous aqueous phase of cosmetic compositions in the form of an oil-in-water emulsion imposes considerable formulation constraints on those skilled in the art.

Until now, these constraints have led those skilled in the art, wishing to incorporate liposomes into cosmetic compositions, to eliminate the surfactants capable of degrading them, or else, in the particular case of emulsions, to use for the oily phase of said emulsions only very apolar oils, for example silicone oils or squalane, in which the amphiphilic lipids constituting the liposomes are highly insoluble.

There exists a real need to develop cosmetic compositions in the form of stable oil-in-water (O/W) emulsions in which the continuous aqueous phase comprises dispersed liposomes, and in which the dispersed oily phase consists of substances that are less apolar, most particularly of triglycerides, which are well liked for cosmetic emulsion formulations in general.

The use of liposomes in alginate gels is known from the prior art.

Authors (Cohen et al., Biochimica et Biophysica Acta, 1063 (1991) 95-102) have studied a system in which unilamellar liposomes, the bilayer of which is in particular made up of palmitoyl phosphatidylcholine, are encapsulated in a matrix of sodium alginate crosslinked with Ca²⁺ ions.

The applicant has demonstrated that the incorporation, into the aqueous phase of an oil-in-water emulsion containing liposomes within the continuous aqueous phase, of a polysaccharide in water-soluble form, and most particularly of an alginate, preferably of alkali metal alginate, exercises a protective role for liposomes placed in the presence of surfactants in the same continuous aqueous phase of said oil-in-water emulsion.

Thus, entirely surprisingly, the applicant has demonstrated that a polysaccharide in water-soluble form, dissolved in the continuous aqueous phase of a liposome emulsion, protects the liposomes against the action of surfactants present in said continuous phase.

An essential object of the invention is therefore to provide oil-in-water emulsions, the continuous aqueous phase of which contains a dispersion of liposomes and comprises at least one non-ionic surfactant, and the oily phase of which is made up of polar lipids or lipids which are not very apolar.

This result is obtained by incorporating a polysaccharide in the dissolved state into the aqueous phase of the cosmetic composition of the invention, which protects the liposomes against the action of surfactants present in this same aqueous phase so as to ensure the stability of the emulsion.

Thus, according to a first subject, the present invention relates to a composition in the form of an emulsion comprising a continuous aqueous phase and a dispersed fatty phase.

According to an essential characteristic of this first subject, the aqueous phase of this composition contains liposomes in the presence of a non-ionic surfactant chosen from the group consisting of fatty alcohol polyethylene glycol ethers and fatty alcohol polypropylene glycol ethers, and mixtures thereof, and of at least one polysaccharide in water-soluble form.

According to a second subject, the invention relates to a method for preparing such an emulsion.

More specifically, this method makes it possible to prepare a stable emulsion containing liposomes dispersed in the aqueous phase of said emulsion, and at least one non-ionic surfactant chosen from the fatty alcohol polyalkylene glycol ethers defined above, said method comprising, on the one hand, preparing an aqueous phase containing said liposomes and at least one polysaccharide in water-soluble form; and on the other hand, preparing a stock emulsion stabilized with said non-ionic surfactant; and mixing said stock emulsion with said aqueous phase containing the liposomes, thereby obtaining said stable emulsion.

Moreover, according to a third subject, the invention relates to a stable dispersion of liposomes which makes it possible in particular to prepare the cosmetic compositions of the first subject.

Other characteristics and advantages of the invention will become apparent in view of the detailed description which follows and of the examples.

The liposomes may be unilamellar or multilamellar, and are preferably multilamellar liposomes.

The liposomes according to the invention have a diameter of approximately 150 to 200 μm, as measured by laser granulometry as a suspension of liposomes or by Transmission Electron Microscopy with preparation of the sample by cryofracture.

According to the invention, the lipids forming the liposomes are amphiphilic lipids, i.e. molecules having a hydrophilic group which may without distinction be ionic or non-ionic, and a lipophilic group.

In the present description and claims, the term “lipid” covers all substances comprising a lipophilic group, the “fatty” carbonaceous chain of which contains more than 5 carbon atoms.

Similarly, for the purpose of the invention, the term “fatty alcohols” is intended to mean alcohols, the carbonaceous chain of which contains at least 5 carbon atoms.

The amphiphilic lipids may be phospholipids, phosphoaminolipids, glycolipids, or mixtures of these lipids. Such substances consist, for example, of an egg or soybean lecithin, a sphingomyelin, a cerebroside or an oxyethylenated polyglyceryl stearate.

The phospholipids may be phosphatidylcholine, phosphatidylserine, phosphatidylethanolamine, phosphatidylglycerol, phosphatidylinositol, or mixtures thereof.

The bilayers of the liposomes preferably consist of phospholipids originating from lecithin, in particular originating from soybean lecithin.

In a preferred embodiment, these phospholipids are a mixture of two types of soybean lecithin, the first type of soybean lecithin being a mixture of phospholipids comprising more than 90% of phosphatidylcholine, the second type being a mixture of phospholipids comprising between 15% and 30% of phosphatidylcholine.

Lecithins in accordance with those used in the liposomes of the invention are, for example, sold by the company Lucas Meyer under the trademarks Emulmetik® 300 and Emulmetik® 930.

The non-ionic surfactant advantageously consists of a mixture of fatty alcohol polyalkylene glycol ethers advantageously having different HLBs.

The non-ionic surfactant is preferably chosen from fatty alcohol polyethylene glycol ethers and mixtures thereof, in particular ethoxylated derivatives of the stearyl alcohol of formula (A) and mixtures thereof.

CH₃(CH₂)₁₇(OCH₂CH₂)ₙOH (A)

In a particularly advantageous embodiment, a mixture of non-ionic surfactants, one being rather of substantially hydrophilic nature and the second being rather of substantially lipophilic nature, is used.

In a preferred embodiment of the invention, the non-ionic surfactant is a mixture of non-ionic surfactants comprising steareth-2, in accordance with formula (A) and in which n=2 on average, sold under the name Brij® 72 and the HLB value of which is 4.9, and steareth-21, in accordance with formula (A) and in which n=21 on average, sold under the name Brij® 721P and the HLB value of which is 15.3.

The steareth-2/steareth-21 ratio is adjusted so as to stabilize the emulsion without degrading the liposomes.

The polysaccharide(s) in water-soluble form may be chosen from a wide range of water-soluble polysaccharides. They may in particular be chosen from the group consisting of starch or a derivative thereof, cellulose derivatives, pectins, gums, alginate, dextrans, carragheenates and hyaluronic acid.

Among the cellulose derivatives, carboxymethylcellulose, hydroxymethylcellulose, cellulose acetate or methylcellulose will in particular be chosen.

Among the gums, xanthan gum or guar gum will in particular be chosen.

However, alginates, in particular alkali metal alginates, and most particularly a sodium or potassium salt or an extract containing same, for example an algal extract, will advantageously be chosen.

In a preferred embodiment, the composition contains at least one alkali metal alginate and at least a second polysaccharide in water-soluble form, in particular an alkali metal salt of carboxymethylcellulose, preferably sodium carboxymethylcellulose.

The polysaccharide concentration will be chosen so as to effectively protect the liposomes against their degradation under the effect of the non-ionic surfactants present in the continuous aqueous phase and intended to stabilize the emulsion.

Preferably, the total amount of water-soluble polysaccharide is between 0.1% and 10% by weight of the composition, preferably between 0.1% and 2% by weight.

Care will also be taken to ensure that the [phospholipids/ water-soluble polysaccharide] ratio of the composition is between 0.1 and 20, preferably between 1 and 10.

The cosmetic composition according to the invention which necessarily comprises at least one water-soluble polysaccharide may also advantageously contain at least one hydrophilic polymer, preferably chosen from the group consisting of polyvinylpyrrolidone and polyvinyl alcohol, and mixtures thereof.

In addition, the cosmetic composition according to the invention may comprise other water-soluble hydrophilic compounds.

The soluble molecules may, for example, be a C₆ or C₁₂ sugar, advantageously chosen from glucose, sorbitol, sucrose, lactitol, glycerol or one of their ethers or esters or of their derivatives.

These water-soluble molecules are preferably obtained from a plant extract.

In one variant of the invention, the cosmetic composition may comprise one or more cosmetically active agents.

Those skilled in the art will understand that the choice of this (or these) cosmetically active agent(s) depends of course on the desired cosmetic properties.

They will also easily understand that, depending on the nature of this agent, it will be present either in the fatty phase or in the aqueous phase, or at least partially incorporated into the liposomes.

Preferably, at least a part of the cosmetically active agents is encapsulated in the liposomes present in the cosmetic composition.

The cosmetically acceptable active agent then becomes concentrated in the hydrophilic or hydrophobic compartments of the liposome according to its own affinity.

Active agents having a strong affinity for lipids are thus preferentially located in the hydrophobic intramembrane spaces, whereas hydrophilic active agents, for example in ionic form, are located in the hydrophilic intermembrane spaces, and in the particular case of liposomal vesicles, also in their core.

The cosmetically active agent encapsulated in the liposomes may advantageously be chosen from the group consisting of substances having a skin-depigmenting activity or a skin-lightening activity; substances having a slimming activity; substances having a hydrating activity; substances having a calming, soothing or relaxing activity; substances having an activity stimulating the microcirculation of the skin, so as to improve the radiance of the complexion, in particular of the face; substances having a sebum-regulating activity for oily skincare; substances for cleansing or purifying the skin; substances having a free-radical scavenger activity or substances having an anti-ageing activity.

The composition according to the invention may advantageously comprise several cosmetically active substances chosen from the same group or else chosen from groups of substances having a different cosmetic effect.

As substance with skin-depigmenting or skin-lightening activity, use is preferably made of a substance chosen from the group consisting of ascorbic acid derivatives, especially esters such as ascorbyl glucosides and ascorbyl phosphates, in particular magnesium ascorbyl phosphate, and an extract of black elder (Sambucus nigra) fruits or flowers.

The substance with skin-depigmenting or skin-lightening activity will advantageously be used at a concentration of between 0.001% and 5%, and in particular between 0.01% and 3% by weight, relative to the total weight of the composition.

As substance with a hydrating effect, use may, for example, be made of glycerol, at least one alcohol such as an alkylene glycol, and in particular propylene glycol, butylene glycol, pentylene glycol and mixtures thereof in any proportions, in particular those commercially available of PEG-60 type. The composition will advantageously contain from 0.001% to 5% by weight, preferably from 0.1% to 5%.

As substance with a calming, soothing or relaxing effect, use may, for example, be made of a glycyrrhizate, in particular in the form of a potassium salt.

As substance with free-radical scavenger or anti-ageing activity, use may, for example, be made of a tocopheryl acetate or tocopheryl phosphate, preferably tocopheryl phosphate.

According to a particular variant embodiment, the proportion by weight of each of these substances will be between 0.001% and 5%.

As substance for reducing wrinkles, use may advantageously be made of the palmitoyl pentapeptide-3 sold under the name (Remixyl®) Matrixyl, in particular (Remixyl) Matrixyl® 3000, an extract of mallow (Vitactyl®), an extract of maize grain (Deliner®, Zea mays kernel extract), or an extract of oat bran (Osilift®, Avena sativa bran extract).

As substance with a slimming effect, use may, for example, be made of xanthine, such as caffeine. According to a particular variant embodiment, the proportion by weight of substance with a slimming effect will be between 0.001% and 5%.

As substance stimulating the microcirculation of the skin, use may, for example, be made of ruscogenin.

According to a particular variant embodiment, the proportion by weight of substance stimulating the microcirculation of the skin will be between 0.001% and 5%.

As substance with sebum-regulating activity for oily skincare, use may, for example, be made of zinc oxide or at least one zinc-based derivative, in particular the organic zinc salts, such as zinc gluconate, zinc salicylate or zinc pidolate.

According to a particular variant embodiment, the proportion by weight of substance with sebum-regulating activity will be between 0.01% and 10%.

According to yet another particular embodiment of the invention, it may be advantageous to add, to said cosmetic composition, at least one refreshing agent such as, for example, menthol or a derivative thereof, such as menthoxypropanediol.

In a preferred embodiment of the invention, the dispersed phase of the invention is a lipid phase which is polar or not very apolar.

In a preferred variant of the invention, the oily phase of said emulsion may comprise relatively polar compounds, in particular triglycerides.

According to a particularly advantageous variant of the invention, the compositions of the invention may be in the form of a multiple emulsion in which the fatty phase of the emulsion is itself a water-in-oil (W/O) emulsion.

Finally, the composition according to the invention may also comprise, in addition, one or more cosmetically acceptable excipients chosen from the group consisting of pigments, dyes, rheological agents, fragrances, sequestering agent, electrolytes, pH adjusters, antioxidants, preservatives, and mixtures thereof, texturing agents, and antisun agents or sunscreens.

The present invention also relates to the method for preparing the cosmetic composition defined above, which method comprises the following steps:
preparing an aqueous phase containing said liposomes and at least one polysaccharide in water-soluble form;
preparing a stock emulsion stabilized with said non-ionic surfactant, and
mixing said stock emulsion with said aqueous phase containing the liposomes, thereby obtaining a stable emulsion.

In a preferred embodiment of the method according to the invention, the polysaccharide is brought into contact with a hydrated lipid phase prior to the step of forming the liposomes by shearing.

Thus, according to a first particularly advantageous variant of this method, it comprises the following successive steps:
preparing a hydrated lamellar lipid phase,
incorporating said hydrated lamellar lipid phase into an aqueous solution comprising at least one water-soluble polysaccharide,
forming the liposomes by shearing the mixture above and
incorporating the mixture above into the stock emulsion containing said non-ionic surfactant.

In the above variant of the method, the preparation of a hydrated lamellar phase is obtained by preparing a concentrated surfactant phase in which the amphiphilic lipids are dissolved in glycerol and 1,3-butylene glycol and then water so as to form a hydrated lamellar phase.

Next, the hydrated lamellar phase previously formed is incorporated into an aqueous solution comprising the polysaccharide(s), vigorously stirred, for example with an Ultra Turrax, so as to form an aqueous dispersion of liposomes.

According to this variant, a fraction of the total water-soluble polysaccharide amount can be incorporated directly into the stock solution.

By applying this first variant of the preparation method, a fraction of the amount of polysaccharides can be encapsulated into said liposomes, the other fraction then being dissolved in the free state, i.e. “non-encapsulated”, in the aqueous phase.

According to a second particularly advantageous variant of this method, it comprises the following successive steps:
preparing a hydrated lamellar lipid phase,
forming the liposomes by shearing the mixture above,
incorporating the dispersion of liposomes into an aqueous solution comprising at least one water-soluble polysaccharide, and
incorporating the mixture above into the stock emulsion containing said non-ionic surfactant.

In the above variant of the method, the preparation of a hydrated lamellar phase is obtained by preparing a concentrated surfactant phase in which the amphiphilic lipids are dissolved in glycerol and 1,3-butylene glycol and then water, so as to form a hydrated lamellar phase.

Next, the hydrated lamellar phase previously formed is vigorously stirred, for example with an Ultra Turrax, so as to form an aqueous dispersion of liposomes.

The dispersion of liposomes is then incorporated into an aqueous solution comprising the alkali metal alginate salt, it being possible for said aqueous solution to be the continuous aqueous phase of the emulsion according to the invention.

By applying this preparation method the entire amount of polysaccharides is dissolved in the free state, i.e. “non-encapsulated”, in the aqueous phase.

The cosmetically active agent, optionally added, may be outside the liposomes in the aqueous phase of the dispersion of liposomes, or may be partially or completely encapsulated in the liposomes.

In an alternative embodiment, the water of the aqueous suspension of liposomes obtained in step 3 can be eliminated by atomizing or freeze-drying so as to obtain a powder comprising the components of the suspension, said powder then being redispersed in the second aqueous phase comprising at least the non-ionic surfactant.

As disclosed above, the invention also relates to a stable dispersion of liposomes. This dispersion can in particular be used for the preparation of the aqueous phase of a composition in the form of emulsions constituting the first subject of the invention. This stable dispersion is obtained by incorporating a hydrated lipid phase into an aqueous solution comprising at least one water-soluble polysaccharide and shearing the mixture obtained, so as to form said dispersion.

### EXAMPLES

### Example 1

### Rich Cream Comprising Liposomes

| | | |
|---|---|---|
| Phase A | Steareth 2 flakes (Brij ® 72 flakes) | 1.3% |
| | Steareth 21 flakes (Brij ® 721P) | 2.2% |
| | 95% cetyl alcohol | 1.2% |
| | Stearyl alcohol | 1.2% |
| | Stearic acid | 0.35% |
| | Palmitic acid | 0.35% |
| | Cetyl palmitate | 1.3% |
| | Hydrogenated polyisobutene | 5.3% |
| | Dicaprylyl carbonate | 4.5% |
| | Caprylic/capric triglycerides | 5% |
| | Dimethicone | 0.2% |
| | Cyclopentasiloxane | 2.1% |
| | Preservatives | 0.7% |
| Phase B | Glycerol | 3.5% |
| | Purified water | 40.6% |
| | Acrylates/C10-C30 alkyl acrylate crosspolymer | 0.5% |
| Phase C | Tetrasodium EDTA | 0.2% |
| | Sodium hydroxide | 0.1% |
| | Caprylyl glycol | 0.5% |
| | Purified water | 4.8% |
| Phase D | Sorbitol | 0.4% |
| | Sodium alginate | 0.2% |
| | Sodium carboxymethylcellulose | <0.1% |
| | Polyvinyl alcohol | <0.1% |
| | Emulmetik ® 300 IP | 0.5% |
| | Emulmetik ® 930 | 0.5% |
| | 1,3-Butylene glycol | 1.0% |
| | Glycerol | 1.0% |
| | Vitamin E phosphate, sodium salt | 0.2% |
| | Antioxidants | 0.2% |
| | Purified water | qs 100% |

The phospholipids of phase D are homogenized with an Ultra Turrax.

The phospholipids are then homogenized with the butylene glycol and the glycerol for 20 minutes and the mixture is left to stand for at least 60 minutes.

The other compounds of phase D are added, followed by the purified water.

The lamellar phase obtained is sheared for 20 minutes with an Ultra Turrax so as to form the dispersion of liposomes.

Phases A and B are heated to 85° C. separately so as to obtain two homogeneous solutions.

Phase B is then emulsified in oily phase A.

The O/W emulsion obtained is gradually cooled with stirring, and then, at 70° C., the compounds of phase C are added, in particular to neutralize the polymers.

The dispersion of liposomes is added to the O/W emulsion previously prepared, with stirring and without incorporating air.

The emulsion obtained comprises multilayer liposomes in the continuous aqueous phase.

These liposomes are not destroyed by the action of the surfactants stabilizing the emulsion.

### Example 2

### Fluid Cream Comprising Liposomes

| | | |
|---|---|---|
| Phase A | Steareth 2 flakes (Brij ® 72 flakes) | 1% |
| | Steareth 21 flakes (Brij ® 721P) | 1% |
| | 95% Cetyl alcohol | 0.3% |
| | Stearyl alcohol | 0.3% |
| | Hydrogenated polyisobutene | 4.3% |
| | Cetearyl isononanoate | 4.2% |
| | Dicaprylyl carbonate | 4.5% |
| | Stearic acid | 0.2% |
| | Palmitic acid | 0.2% |
| | Cetyl palmitate | 1.3% |
| | Dimethicone | 0.3% |
| | Preservatives | 0.7% |
| Phase B | Purified water | 46.7% |
| | Glycerol | 6.0% |
| | Sorbitol | 0.2% |
| | Sodium alginate | 0.1% |
| | Sodium carboxymethylcellulose | <0.1% |
| | Polyvinyl alcohol | <0.1% |
| | Acrylates/C10-C30 alkyl acrylate crosspolymer | 0.6% |
| | Methyl methacrylate crosspolymer | 1.5% |
| | 1,3-Butylene glycol | 2.0% |
| Phase C | tetrasodium EDTA | 0.2% |
| | Sodium hydroxide | 0.1% |
| | Caprylyl glycol | 0.5% |
| | Purified water | 2.1% |
| Phase D | Sorbitol | 0.2% |
| | Alginate | 0.1% |
| | Sodium carboxymethylcellulose | <0.1% |
| | Polyvinyl alcohol | <0.1% |
| | Emulmetik ® 300 IP | 0.5% |
| | Emulmetik ® 930 | 0.5% |
| | 1,3-Butylene glycol | 1.0% |
| | Glycerol | 1.0% |
| | Vitamin E phosphate | 0.2% |
| | Antioxidants | 0.2% |
| | Purified water | qs 100% |

The method is in accordance with Example 1

The product obtained is a light cream in the form of an emulsion.

1. Composition in the form of an emulsion comprising a continuous aqueous phase and a dispersed fatty phase, characterized in that said aqueous phase contains liposomes in the presence of a non-ionic surfactant chosen from fatty alcohol polyethylene glycol ethers and fatty alcohol polypropylene glycol ethers, and mixtures thereof, and of at least one polysaccharide in water-soluble form.

2. Composition according to claim 1, characterized in that said liposomes are multilamellar liposomes.

3. Composition according to claim 1, characterized in that the liposomes are made up of amphiphilic lipids chosen from the group consisting of phospholipids, phosphoaminolipids, glycolipids and mixtures thereof.

4. Composition according to claim 3, characterized in that said phospholipids are chosen from the group consisting of phosphatidylcholine, phosphatidylserine, phosphatidylethanolamine, phosphatidylglycerol, phosphatidylinositol, and mixtures thereof.

5. Composition according to claim 4, characterized in that said mixture of phospholipids is a mixture of two types of soybean lecithin, the first type of soybean lecithin being a mixture of phospholipids comprising more than 90% of phosphatidylcholine, the second type being a mixture of phospholipids comprising between 15% and 30% of phosphatidylcholine.

6. Composition according to claim 1, characterized in that the liposomes are made up of amphiphilic lipids chosen from the group consisting of egg lecithin, soybean lecithin, sphingomyelin, cerebrosides and oxyethylenated polyglyceryl stearates.

7. Composition according to claim 1, characterized in that it contains a mixture of non-ionic surfactants chosen from fatty alcohol polyethylene glycol ethers and fatty alcohol polypropylene glycol ethers.

8. Composition according to claim 1, characterized in that said non-ionic surfactant is chosen from fatty alcohol polyethylene glycol ethers and mixtures thereof.

9. Composition according to claim 1, characterized in that the non-ionic surfactant is chosen from stearyl alcohol polyethylene glycol ethers.

10. Composition according to claim 9, characterized in that said non-ionic surfactant is a mixture of steareth-2 and steareth-21.

11. Composition according to claim 1, characterized in that said polysaccharide in water-soluble form is chosen from the group of water-soluble polysaccharides consisting of starch or a derivative thereof, cellulose derivatives, pectins, gums, alginate, dextrans, carragheenates and hyaluronic acid.

12. Composition according to claim 1, characterized in that said polysaccharide in water-soluble form is an alkali metal alginate.

13. Composition according to claim 1, characterized in that said composition contains at least one alginate in water-soluble form, and a second polysaccharide in water-soluble form as defined in claim 10 said non-ionic surfactant is a mixture of steareth-2 and steareth-21.

14. Composition according to claim 13, characterized in that the second polysaccharide is an alkali metal salt of carboxymethylcellulose.

15. Composition according to one of claims 1, characterized in that the total amount of polysaccharide in water-soluble form is effective for protecting the liposomes against their degradation under the effect of said surfactant(s).

16. Cosmetic composition according to claim 1, characterized in that the total amount of water-soluble polysaccharide is between 0.1% and 10% by weight of the composition

17. Cosmetic composition according to claim 1, characterized in that the [phospholipids/water-soluble polysaccharide] ratio of the composition is between 0.1 and 20.

18. Composition according to claim 1, characterized in that it also contains at least one hydrophilic polymer.

19. Composition according to claim 18, characterized in that said hydrophilic polymer is chosen from the group consisting of polyvinylpyrrolidone and polyvinyl alcohol, and mixtures thereof.

20. Cosmetic composition according to claim 1, characterized in that it also contains at least one water-soluble hydrophilic compound chosen from the group consisting of C₆ or C₁₂ sugars, ethers thereof, esters thereof and derivatives thereof.

21. Composition according to claim 1, characterized in that the fatty phase of said emulsion contains triglycerides.

22. Composition according to one of claims 1, characterized in that the fatty phase of said emulsion is itself a water-in-oil (W/O) emulsion.

23. Cosmetic composition according to claim 1, characterized in that it contains at least one cosmetically active agent contained in the aqueous phase and/or the fatty phase of the emulsion, and at least one cosmetically acceptable excipient.

24. Cosmetic composition according to claim 1, characterized in that at least one active agent is at least partially incorporated into the liposomes.

25. Cosmetic composition according to claim 1, characterized in that the active agent(s) is (are) chosen from the group consisting of substances having a skin-depigmenting activity or a skin-lightening activity; substances having a slimming activity; substances having a hydrating activity; substances having a calming, soothing or relaxing activity; substances having an activity stimulating the microcirculation of the skin so as to improve the radiance of the complexion, in particular of the face; substances having a sebum-regulating activity for oily skincare; substances for cleansing or purifying the skin; substances having a free-radical scavenger activity or substances having an anti-ageing activity.

26. Method for preparing a composition according to claim 1, characterized in that it comprises the following steps:
preparing an aqueous phase containing said liposomes and at least one polysaccharide in water-soluble form,
preparing a stock emulsion stabilized with said non-ionic surfactant, and
mixing said stock emulsion with said aqueous phase containing the liposomes, thereby obtaining said stable emulsion.

27. Method according to claim 26, characterized in that it comprises the following successive steps:
preparing a hydrated lamellar lipid phase,
incorporating said hydrated lamellar lipid phase into an aqueous solution comprising at least one water-soluble polysaccharide,
forming the liposomes by shearing the mixture above, and
incorporating the mixture above into the stock emulsion containing said non-ionic surfactant.

28. Method according to claim 27, characterized in that a fraction of the total amount of water-soluble polysaccharide is directly incorporated into the stock solution.

29. Method according to claim 26, characterized in that it comprises the following successive steps:
preparing a hydrated lamellar lipid phase,
forming the liposomes by shearing the mixture above,
incorporating the dispersion of liposomes into an aqueous solution comprising at least one water-soluble polysaccharide, and
incorporating the mixture above into the stock emulsion containing said non-ionic surfactant.

30. Stable dispersion of liposomes that can be used for the preparation of the aqueous phase of a composition as defined in claim 1, characterized in that it is obtained by incorporating a hydrated lipid phase into an aqueous solution comprising at least one water-soluble polysaccharide and shearing the mixture obtained, so as to form said dispersion.
